(19) United States
(12) Patent Application Publication
(10) Pub. No.: US 2024/0228571 A9
(43) Pub. Date: Jul. 11, 2024

(54) PROCESS OF PRODUCTION AND PURIFICATION OF HYBRID OR NON-HYBRID RECOMBINANT GLYCOPROTEIN HORMONES, HYBRID OR NON-HYBRID RECOMBINANT GLYCOPROTEIN HORMONES, EXPRESSION VECTORS AND USES OF RECOMBINANT GLYCOPROTEIN HORMONES

(71) Applicants: UNIVERSIDADE DE SÃO PAULO - USP, Butantä (BR); LCR KIMERA BIOTECNOLOGIA LTDA - ME, Riberiräo Preto (BR)

(72) Inventors: Marcelo Dias BARUFFI, Ribeirão Preto (BR); Camillo del Cistia ANDRADE, Ribeirão Preto (BR); Rubens Eduardo da SILVA, Ribeirão Preto (BR); Robinson Antonio Martins de OLIVEIRA, Ribeirão Preto (BR); Daniel Roberto CALLEJON, Ribeirão Preto (BR)

(21) Appl. No.: 18/375,407

(22) Filed: Sep. 29, 2023

Related U.S. Application Data
(63) Continuation-in-part of application No. 16/086,284, filed on Sep. 18, 2018, now abandoned.

(30) Foreign Application Priority Data
Mar. 22, 2016 (BR) 10 2016 006222 5

(51) Int. Cl. C07K 14/59; A61K 38/00; A61P 15/08; C07K 1/22; C07K 1/34

(52) U.S. Cl. CPC C07K 14/59; A61P 15/08; C07K 1/22; C07K 1/34; A61K 38/00

(57) ABSTRACT

Disclosed is a method for producing hybrid or non-hybrid recombinant glycoprotein hormones, for example the recombinant equine chorionic gonadotropin (r-eCG), the hybrid recombinant chorionic gonadotropin, the recombinant thyroid-stimulating hormone (r-TSH), the recombinant luteinizing hormone (r-LH), the luteinizing hormone and the recombinant follicle-stimulating hormone (r-FSH). In addition, the present disclosure relates to the recombinant glycoprotein hormones comprising the equine α and β subunits, inter alia, the α subunit of mammals and equine β subunit, where the two subunits are fused in a simple chain, and chain-modifying agents, which hormones are easier to purify, more homogeneous, easier to produce on an industrial scale without using animals, in comparison with the wild glycoprotein hormone. The hormones are useful for inducing animal reproduction, ovulation induction, superovulation induction, follicle growth, estrus induction, anoestrus reversal, puberty induction in animals, both with and without commercial interest.

### CROSS REFERENCE TO RELATED APPLICATIONS

This application is a continuation-in-part application of U.S. patent application Ser. No. 16/086,284, filed Sep. 18, 2018, which in turn is a 371 Application of PCT International Patent Application Serial No. PCT/BR2016/000051, filed May 10, 2016, the contents of which are disclosed herein.

### FIELD OF THE INVENTION

The present invention belongs to the field of processes for producing peptide hormones; specifically, it belongs to the field of processes for producing peptide hormones containing more than 20 amino acids; and describes a process for producing and purifying hybrid or non-hybrid recombinant glycoprotein hormones, hybrid or non-hybrid recombinant glycoprotein hormones, including their expression vectors, and uses thereof.

### REFERENCE TO AN ELECTRONIC SEQUENCE LISTING

The contents of the electronic sequence listing (sequencelisting.xml; Size: 144,988 bytes; and Date of Creation: Jan. 16, 2024) is herein incorporated by reference in its entirety.

### BACKGROUND OF THE INVENTION

In recent years, numerous biotechnological processes of production and purification of protein and glycoprotein hormones have been developed. All processes developed until then have their own strategies that vary according to the hormone to be produced and that aim at increasing the production of the hormone, or are aimed at facilitating the purification step.

In the production of recombinant glycoproteins, the state of the art uses mammalian cells due to their ability to promote the correct folding and post-translational processing. Several factors are involved in the optimization of protein expression in mammalian cells. One of these factors is the expression vectors for generation of recombinant cell lines using strong promoters of viral or cellular origin, such as the cytomegalovirus (CMV) promoter (Gopalkrishnan et al., 1999). Currently, most of the high protein production processes for the Biopharmaceutical industry (about 60-70%) are based on cells grown in suspension (Moritz, et al, 2015).

Equine Chorionic Gonadotropin (eCG) is a glycoprotein hormone produced in the trophoblast of pregnant mares, consisting of 2 subunits (a and β), with similar action on the follicle stimulating hormone (FSH) and luteinizing hormone (LH), both from the hypophysis and with important action in the events of induction of follicular growth and luteinization, respectively (Murphy, 2012). This bi-functional hormonal activity occurs after the administration of eCG in species of mammals other than horses, such as cattle, swine, sheep and goats (Murphy, 2012). The N-glycosylation sites of the alpha chain of the eCG is fundamental for the expression of its LH activity (Min et al. 1996; Min et al., 2004; Bousfield et al., 2004; Murphy, 2012). The loop region of the eCG protein structure and a sequence of amino acid residues (104-109) of the C-terminal region of the beta chain of this hormone are associated with the bi-functional action of eCG (LH and FSH activity) and its FSH function, respectively (Moyle et al. 1994; Galet et al. 2009).

eCG is used in different protocols of assisted animal reproduction. The use of eCG in other mammals may induce the production of anti-eCG antibodies and they may decrease the biological actions of this hormone in these animals (Herve et al. 2004, Forcada et al. 2011). The alpha chain of the eCG molecule is the major antigenic portion of this hormone (Chopineau et al, 1993).

The eCG gene is present on chromosome 10 in Equus caballus, and its expression generates the subunits, (i) gonadotropin alpha 1 subunit (chr10: 39937900-39940069; Gene ID: 100034174), its transcription undergoes splicing of 3 exons generating a messenger (mRNA) of nearly 2 Kb and an open reading frame (ORF) of 363 nucleotides translated into a mature protein of 120 amino acids and approximately 13.8 kDa, and (ii) chorionogonadotropin subunit beta (chr10: 18963366-18964444; Gene ID: 100054774), its transcription undergoes splicing of 3 exons generating a mRNA of approximately 520 kb and its open reading frame (ORF) of 510 nucleotides is translated into a mature protein of 169 amino acids and approximately 17.8 kDa (http://genome.ucsc.edu, http://www.ncbi.nlm.nih.gov).

The CGA gene of the Bos taurus species is present on chromosome 9 at position chr9: 63692501-63694585, and its expression generates the subunit, gonadotropin alpha 1 subunit (Gene ID: 280749), its transcription undergoes splicing of 4 exons generating a mRNA of approximately 742 base pairs (bp) (provisional to date) and open reading frame (ORF) of 363 nucleotides translated into a mature protein of 120 amino acids and approximately 13.6 kDa (http://genome.ucsc.edu, http://www.ncbi.nlm.nih.gov).

The CGA gene of the Sus scrofa species is present on chromosome 10 at position chr10: 62246069-62248001, and its expression generates the subunit, gonadotropin alpha 1 subunit (Gene ID: 406869), its transcription undergoes splicing of 3 exons generating a mRNA of approximately 363 base pairs (bp) (provisional to date) and open reading frame (ORF) of 363 nucleotides translated into a mature protein of 120 amino acids and approximately 13.5 kDa (http://genome.ucsc.edu, http://www.ncbi.nlm.nih.gov).

The CGA gene of the Ovis aries species is present on chromosome 8 at position chr8: 49919904-49921988, and its expression generates the subunit, gonadotropin alpha 1 subunit (Gene ID: 443538), its transcription undergoes splicing of 4 exons generating a mRNA of approximately 716 base pairs (bp) (provisional to date) and open reading frame (ORF) of 363 nucleotides translated into a mature protein of 120 amino acids and approximately 13.5 kDa (http://genome.ucsc.edu, http://www.ncbi.nlm.nih.gov).

The CGA gene of the Capra hircus species is present on chromosome 8 at position chr8: 49919901-49921988, and its expression generates the subunit, gonadotropin alpha 1 subunit (Gene ID: 100860817), its transcription undergoes splicing of 3 exons generating a mRNA of approximately 366 base pairs (bp) (provisional to date) and open reading frame (ORF) of 363 nucleotides translated into a mature protein of 120 amino acids and approximately 13.5 kDa (http://genome.ucsc.edu, http://www.ncbi.nlm.nih.gov).

### STATE OF THE ART

Several patent documents relate to processes of producing chorionic gonadotrophin from equine and other mammals. For example:

Document EP0974599 discloses a recombinant equine chorionic gonadotropin hormone in which the α and β chains of equine chorionic gonadotropin are bonded. This patent also claims the veterinary uses of this recombinant molecule.

On the other hand, document PI 0108556-5 describes a purification process of recombinant human Chorionic Gonadotropin (rhCG) produced in CHO cell cultures, which comprises the combined use of ion exchange chromatography and reverse phase HPLC. Such document also claims a pharmaceutical composition containing rhCG for subcutaneous administration.

Documents PI 9814880-0 and PI 9914670-3 relate to single-chain recombinant glycoprotein hormones, the process of producing the same without the use of a purification system by affinity chromatography, of fluorescent label and a polypeptide characteristic of cell secretion.

Document U.S. Pat. No. 5,260,421 discloses a method of promoting site-directed mutagenesis in glycoproteins in general, for the production of hormones, such as luteinizing hormone, follicle stimulating hormone, thyroid stimulating hormone, and chorionic gonadotrophin. Document U.S. Pat. No. 6,469,139 discloses a modified human chorionic gonadotrophin at specific sites of the amino acid sequence, and its medical use as an immunological contraceptive.

Document WO9532216 discloses a method of producing biologically active glycoprotein hormones in prokaryotic cells which employs a redox thiol buffer to form structurally active subunits of the hormone.

Documents JPH1036398 and JPH1036399 relate to processes of producing recombinant equine chorionic gonadotropin, in which the subunits are not fused in a single-chain. The difference between them is due to the fact that the former claims the use of r-eCG in AI procedures or superovulation in cattle, while the latter claims the activity of stimulating the production of FSH.

Document WO2014183175 discloses methods for the production and purification of follicle stimulating hormone (FSH) using a parent or mutant HEK 293 cell platform.

Thus, no reports were found in the state of the art concerning the methods of obtaining and using artificial insemination and superovulation protocols related to the hybrid forms of chorionic gonadotrophin composed by the association of non-equine alpha chains and equine beta chains.

### SUMMARY OF THE INVENTION

The present invention aims to propose a process for producing and purifying hybrid or non-hybrid recombinant glycoprotein hormones.

In addition, the present invention proposes recombinant glycoprotein hormones of equine origin (r-eCG) and other hybrids containing portions of equine (β chain) and target mammalian origin (α chain), resulting in chimeric glycoprotein hormones specific to the target species, aiming at obtaining a hormonal composition that possesses the LH and FSH activities and without immunotoxicity to the target species.

In addition, the present invention further proposes the use of hybrid and non-hybrid recombinant glycoprotein hormones obtained with the use of their expression vectors and their pharmaceutical compositions in assisted animal reproduction of target species of commercial or non-commercial interest, mammals in general, like cattle, sheep, goats, pigs, horses, mules, buffaloes, bison, antelopes, domestic and wild species of canines and felines, cetaceans, ursids and primates.

### BRIEF DESCRIPTION OF DRAWINGS

FIG. 1 shows the electrophoretic analysis of the amplification procedure of the gene fragment referring to SEQ. ID. 16;

FIG. 2 shows the electrophoretic analysis of cleavage products of recombinant clones (SEQ. ID. 16) of SEQ. ID. 38;

FIG. 3 shows the electrophoretic analysis of cleavage products of recombinant clones (SEQ. ID. 16) of SEQ. ID. 39;

FIG. 4 shows the expression analysis of expression of the GFP molecule in CHO-K1 cells transfected with SEQ. ID. 39, by fluorescence microscopy, where (A) and (C) illustrate DIC (Differential Interference Contrast) images and show a similar cell growth pattern between the two cell populations, while (B) and (D) illustrate the fluorescence related to the presence of the GFP protein;

FIG. 5 shows the electrophoretic analysis of cell culture supernatant containing the SEQ. ID. 17 (non-fused recombinant r-eGG to GFP molecule); in which it is possible to observe in MM—Molecular Marker; MC—Culture medium (Freestyle Serum Free, C—HEK 293 cells after 48 hour culture; SB—Supernatant of HEK 293 cell culture after 48 hour culture, where it is possible to observe the SEQ. ID. 17 band;

FIG. 6 shows the electrophoretic analysis of purified preparation of SEQ. ID. 19. 12% SDS-PAGE, where it is possible to observe in MM—Molecular Marker; C—Culture Medium (DMEM containing 10% fetal bovine serum); S+—Cell culture supernatant; FT—Flow Through, proteins that did not bind to the His-Trap column; E1 to 5—Fractions eluted from the column, where it is possible to observe the band referring to the SEQ. ID. 19;

FIG. 7 shows purified preparations of SEQ. ID. 19 inducing the release of estradiol and progesterone in the serum of rats;

FIG. 8 shows the analysis of in vivo activity of SEQ. ID. 17 corresponding to non-fused recombinant eCG to GFP and purified from culture of the supernatant of HEK 293 cells cultured in the absence of fetal bovine serum using the Freestyle Serum Free culture medium;

FIG. 9 shows the functional comparative analysis between the recombinant forms of the control molecule (SEQ. ID: 49), of native eCG and SEQ. ID. 19, where (A) represents images of the ovaries of intramuscularly treated prepubertal rats with PBS (negative control), SEQ. ID. 49, native eCG and SEQ. ID. 19;

FIG. 10 shows the comparative analysis of the pregnancy rate in females, for the evaluation of the activity of SEQ. ID. 17 in large animals (cattle). Graphic representation of the percentage of pregnancy rate of females induced to estrus through hormonal protocols performed by the administration of eCG 300 IU and of SEQ. ID. 17 30 μg. The analysis was performed by ultrasonography after 30 days of insemination of Bos indicus females, with homogeneous groups for the animal category (race, age, calving at least 1 time), with n=127 for the eCG group and n=50 for the group SEQ. ID. 17 group.

FIG. 11 shows the graphic representation of vectors relating to SEQ. ID. 38 and SEQ. ID. 39, which represent all the vectors described in this invention.

FIG. 12 shows the organization chart of the steps of the production and purification process of recombinant glycoprotein hormones of this invention.

### DETAILED DESCRIPTION OF THE INVENTION

The present invention relates to a process for the production and purification of recombinant glycoprotein hormones comprising the steps of:
(a) amplification, modification and cloning of the hybrid or non-hybrid molecules;
(b) construction of the expression vectors of recombinant glycoprotein hormones;
(c) transfection, expression and analysis of cells expressing the recombinant glycoprotein hormones;
(d) purification of recombinant glycoprotein hormones by affinity chromatography;
(e) dialysis and sterilization of recombinant glycoprotein hormones.

Recombinant glycoprotein hormone (r-eCG), whether or not fused to the GFP molecule, and its hybrid forms of the present invention, are selected from the group consisting of recombinant equine chorionic gonadotrophin (r-eCG), recombinant bovine chorionic gonadotrophin (r-bCG), recombinant swine chorionic gonadotrophin (r-sCG), recombinant sheep chorionic gonadotrophin (r-oCG), recombinant goat chorionic gonadotrophin (r-cCG), recombinant thyroid stimulating hormone (r-TSH), recombinant luteinizing hormone (r- and recombinant follicle stimulating hormone (r-FSH). Preferably, the glycoprotein hormone eCG, fused or not to the GFP molecule, and its obtained hybrid forms represent, respectively, the nucleotide and glycoprotein corresponding to recombinant equine chorionic gonadotrophin (r-eCG) and their hybrid forms.
(a) amplification, modification and cloning of the hybrid or non-hybrid molecules;

The step of PCR amplification of the r-eCG gene fragments (SEQ.ID. 16) or r-eCG-GFP (SEQ.ID. 18) by PCR (Mullis et al., 1986) comprises the use of primer oligonucleotides of SEQ. ID. 1, SEQ. ID. 2 and SEQ. ID. 3 complementary to the different forms of native chorionic gonadotrophin to obtain a gene fragment relating to the fusion between the beta subunit DNA sequence of the native eCG (SEQ. ID. 6) and the native eCG alpha subunit DNA sequence (SEQ. ID. 4), wherein the SEQ. ID. 6 and 4 correspond to the α and β subunits of eCG and additional sequences corresponding to their total in SEQ ID. 16 or SEQ. ID. 18 validated by agarose gel electrophoresis.

SEQ ID. 1, SEQ. ID. 2 and SEQ. ID. 3, in addition to promoting the amplification of the genes related to eCG subunits α and β subunits, present additional nucleotide sequences associated with restriction enzyme cleavage sites and coding sequences for a histidine tail for poly-histidine sequence translation (6× His-Tag), and a proteolytic site, such as the Tobacco Etch Virus (TEV-Tag) protease site, associated with cloning of the gene sequences, purification of recombinant hormones and protein editing of these hormones respectively, thereby generating a fragment of DNA with 847 bp.

Amplification occurs by PCR, the polynucleotide of SEQ. ID. 16 being obtained, which is then purified on chelating resin (Sambrook et al, 1989).
(b) Construction of vectors for the expression of recombinant glycoprotein hormones in eukaryotic cells (CHO-K1 and HEK 293)

The construction of vectors for the expression of recombinant glycoprotein hormones in eukaryotic cells (CHO-K1 and HEK 293) is initiated by the cloning of SEQ. ID. 16 or SEQ. ID. 18 in prokaryotic cells (E. coli DH5a). This cloning step begins with the insertion of the sequences SEQ. ID. 16 or SEQ. ID. 18 in a commercial cloning vector that is used to transform DH5a competent cells by thermal shock. Finally, the selection of bacterial recombinant clones containing the recombined cloning vector is performed with the sequences SEQ. ID. 16 or SEQ. ID. 18, whose presences are validated by agarose gel electrophoresis and DNA chemical sequencing. It should be clear for a person skilled in the art that various techniques and reagents may be used without the difference between the techniques and reagents being able to generate significant differences in the final process. In the present invention, the transformation of competent E. coli DH5a prokaryotes is accomplished by the introduction of cloning vectors by thermal shock, and the selection of recombinant clones is performed by cleavage for the detection of SEQ. ID. 16 and confirmation of the sequence by a chemical method of nucleotide sequencing, described in the literature (Sanger et al, 1997).
(c) Transfection, expression and analysis of cells that produce recombinant glycoprotein hormones.

Expression vectors obtained after the cloning step of the hybrid and non-hybrid hormones are then used to transiently transfect eukaryotic cells with the aid of liposomes. Alternatively, the recombinant hormone gene sequences used in the composition of the expression vectors for unstable eukaryotic cell transformation may be used in the construction of expression vectors for stable transformation of these cells with the use of sequences. SEQ. ID. 16 or SEQ. ID. 18, via lentiviral vectors or biologically safe systems, of non-random gene integration and without the need for selective agents (antibiotics and other chemical substances) such as Transcription Activator-Like Effector Nucleases (TALENs), Clustered Regularly Interspaced Short Palindromic Repeats (CRISPR) and other systems with these properties, for the generation of expression cell lines and their analysis by fluorescence microscopy for visualization of the protein expression of SEQ. ID. 19 and/or by other methodologies (immunodetection or gene sequencing) for detecting expression of the protein of SEQ. ID. 17 and SEQ. ID. 19. On the other hand, stable integration systems such as the use of lentiviral vectors can be used for these same purposes.

It is possible to detect, in the fluorescence microscope analysis, the change in fluorescence of the cells of the culture medium, since SEQ. ID. 19 is exported to culture medium due to its signaling peptide of SEQ. ID. 21, present between amino acids 1-20 of SEQ. ID. 19. In control cells transfected with SEQ. ID. 48, the presence of fluorescence is only detectable in the cell cytoplasm.
(d) Purification of recombinant glycoprotein hormones

Purification occurs by collecting the supernatant from the culture medium of mammalian cells transfected with the sequences SEQ. ID. 16 or SEQ. ID. 18 and that secret the SEQ. ID. 17 or SEQ. ID. 19, transiently or stably, followed by centrifugation between 1200 and 1800 g between 7 and 15 minutes at 4° C. for the removal of cells in suspension and subsequent purification of SEQ. ID 17 or SEQ. ID. 19 by affinity chromatography on nickel resins and after elution, an approximate yield of 15 to 17 mg of purified hormone is obtained for each liter of culture.
(e) Dialysis and sterilization of recombinant glycoprotein hormones.

The final step to obtain the recombinant glycoprotein hormones is carried out by dialysis in concentrators and/or by tangential centrifugation (cut off of 10 to 60 kDa), where the buffer is used in the production and purification steps of glycoprotein hormones in PBS pH 7.4 is changed, followed by sterilization of the solution containing the hormones produced with filters (0.22 μm) suitable for the final volume.

The invention also relates to a recombinant glycoprotein hormone comprising the subunits α and β fused in a single chain and chain modifier agents at the amino and carboxy terminal moieties, such as a fusion site to a fluorescein, such as GFP; a purification marker, such as a poly-His tail; peptide signaling the secretion of the molecule; dimerization interface peptide and a specific proteolytic site, such as the proteolytic cleavage site with the TEV (Tobacco etch virus) proteolytic enzyme, and optionally, a fluorescent label. These modifications provide the recombinant molecule with features not presented by wild-type hormones, such as nickel affinity, alpha- and beta-strand fusion and/or fluorescence emission, which favor its production and purification processes.

All cloning, expression and purification steps described in steps (a) to (d) related the preparation of r-eCG were efficient and effective and should also be used in the cloning, expression and purification steps of the hybrid forms of these recombinant glycoprotein hormones.

Preferably, this production process is used for the production of SEQ. ID. 17 and SEQ. ID. 19, referring to recombinant equine chorionic gonadotrophin which, in vivo, had a bioactivity of approximately 10,000 IU/mg and close to the bioactivity of native eCG preparations.

Aiming at a reduction of the immunogenicity of SEQ. ID. 17 and SEQ. ID. 19 for other species, this invention aimed at obtaining hybrid forms of these glycoprotein hormones composed of the alpha chain of the target species and the equine beta chain with and/or without fusion with the GFP molecule.

Examples for obtaining functional analysis of r-eCG

### Example 1—Construction of the Cloning Vector of SEQ. ID. 16

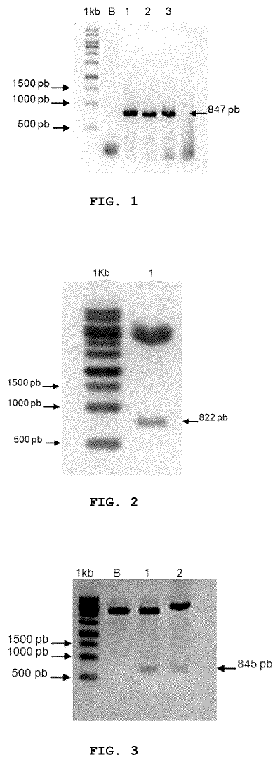

The elaborated gene fragment related to SEQ. ID. 16 was commercially synthesized and amplified by PCR (FIG. 1), using the oligonucleotides (SEQ. ID. 1, SEQ ID. 2 and SEQ. ID. 3). The electrophoretic analysis of the amplification procedure was performed on agarose gel (1%) and with the use of a 1 Kb molecular size marker. The samples tested were: negative control of the PCR reaction (lane B); and gene fragment (SEQ. ID. 16) amplified of approximately 847 bp (lanes 1, 2 and 3).

SEQ. ID. 16, was cloned into a plasmid vector (CloneJet-Thermo) and used to transform W5a competent E. coli cells by heat shock, and, after selection of recombinant clones by cleavage, the sequence confirmation of SEQ. ID., 16 was performed by a chemical nucleotide sequencing method.

### Example 2—Construction of the Expression Vectors of SEQ. ID. 38 and SEQ. ID. 39

Once the SEQ. ID. 16 was confirmed, the recombinant clones were cleaved with the XhoI and EcoRI enzymes, for removing the fragment of the SEQ ID. 16, and cloned for generating SEQ. ID. 38 and SEQ. ID. 39. Selection of recombinant clones was done by cleavage of SEQ. ID. 38 and SEQ. ID. 39. The electrophoresis of clone cleavage products was done on agarose gel (1%) using a 1 Kb molecular marker. As seen in FIG. 2, the clone after cleavage with XhoI and EcoRI is found in lane 1, where the band relative to SEQ. ID. 16 is seen. In FIG. 3, the samples tested were: negative control of the PCR reaction (lane B); where the clones after cleavage of SEQ. ID. 39 are in lanes 1 and 2), where the band referring to SEQ. ID. 16 is seen. Confirmation of the perfect sequence of SEQ. ID. 16 was carried out by chemical DNA sequencing.

### Example 3—Transfection of Mammalian Cells

For the generation of expression cell lines (HEK 293 and CHO-K1), 6-well 500 μL plates (24-well plates) were used for transfecting 800 ng of SEQ. ID. 38 and SEQ. ID. 39, using 2 μL of lipofectamine 2000 (Thermo). As a control, cells were transfected under the same conditions with SEQ. ID. 48. Cells were cultured on Freestyle Serurm Free (Thermo) or DMEM medium (Sigma) containing 10% fetal bovine serum and 1× antibiotic/antimycotic solution for 24 hours for further addition of 400 μg/mL geneticin (G418, Sigma-Aldrich).

### Example 4—Selection of Transfected Mammalian Cell Clones

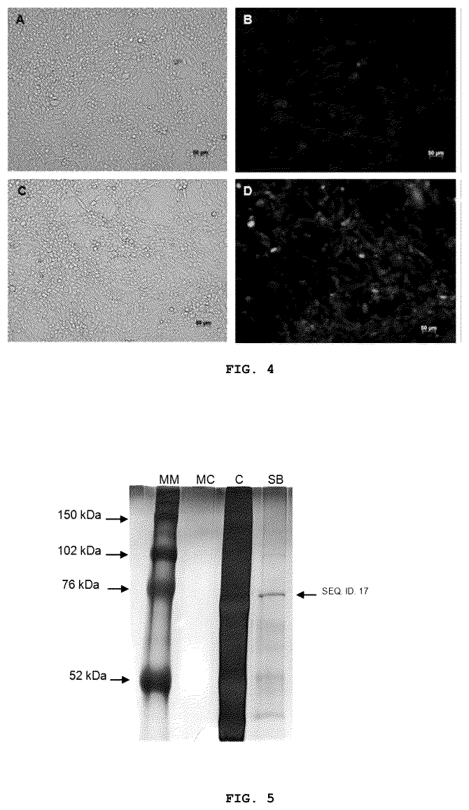

Transfected cells were selected over a period of 3 weeks, with geneticin concentration (G418) changes for elimination of non-transfected clones. Cells were then analyzed using fluorescence microscopy for the expression of SEQ. ID. 19 and of SEQ. ID. 49. The analysis was made by observing the change in the fluorescence of the cells and the culture medium, since SEQ. ID. 19 is exported to the medium due to its signal peptide present between amino acids 1-20 (SEQ. ID. 21). The control cells showed the presence of fluorescence only within the cells, due to the expression of SEQ. ID. 49 (FIG. 4).

Cells transfected with SEQ. ID. 39 express and export SEQ. ID. 19 to the culture medium (FIG. 4 (B)). Cells transfected with SEQ. ID. 48 express the GFP protein (SEQ. ID. 49) in the cells (FIG. 4 (D)).

### Example 5—Purification and Electrophoretic Analysis of SEQ. ID. 17 and SEQ. ID. 19

The selected HEK 293 cells were transferred to Spinner with 100 mL of Freestyle (Thermo) culture medium containing 400 mg/mL geneticin for propagation, increasing the number of cells in the highest culture volume and consequently the concentration of recombinant protein expressed for 96 hours.

The selected CHO-K1 cells were transferred to 75 cm 2 culture bottles containing DEMEN (Sigma) containing 10% fetal bovine serum and geneticin 400 mg/mL for propagation, increasing the number of cells in the highest culture volume and consequently the concentration of recombinant protein expressed for 8 days of propagation with collections of supernatant (culture medium) every 48 hours.

After culture of the HEK 293 and CHO-K1 cells, centrifugation (1500×g/10 min./4° C.) of the culture media was carried out for the removal of cells in suspension, concentration and dialysis in appropriate concentrators, and for further purification of SEQ. ID. 17 and SEQ. ID. 19 by His-Trap column affinity chromatography on a suitable chromatograph. After elution with imidazole gradient (Sigma) (5 to 500 mM), the fractions were analyzed on 12% SDS-PAGE, as seen in FIG. 6. After concentration of the eluted fractions, quantification by absorbance measurement at 280 nm and correction by the correction factor (calculated by the molar extinction coefficient) of 1.29 for SEQ. ID. 17 e 1.34 for SEQ. ID. 19, an approximate yield of 15 mg of purified SEQ. ID. 17 and 17 mg of purified SEQ. ID. 19 for each liter of culture was estimated.

### Example 6—Verification of the Ability of SEQ. ID. 19 to Induce the Production of Hormones In Vivo

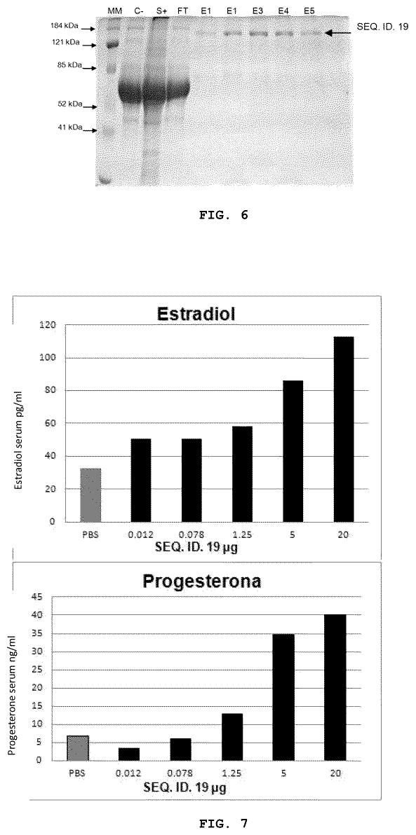

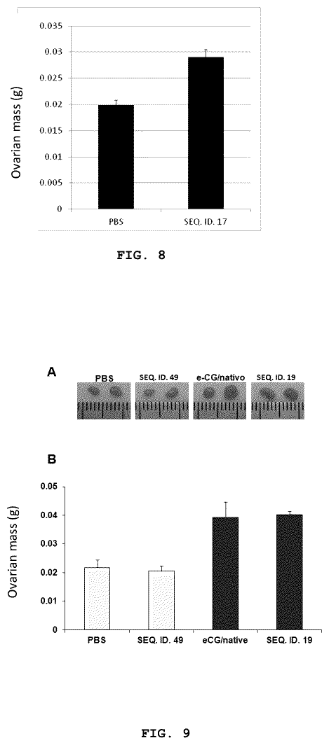

The hormonal effects of SEQ. ID. 19 were evaluated by rat assays and quantified by the chemiluminescence technique. FIG. 7 shows the induction of the production of estradiol (1713-estradiol) and progesterone measured in the serum of immature (Wistar) rats at 4-6 weeks of age after 6 and 18 hours, respectively, of the intramuscular injection of decreasing doses of SEQ. ID. 19 (0.012 to 20 μg), using as a control 4-6 week old immature rats injected with phosphate buffered saline, pH=7.4 (PBS); the procedures used in the hormone induction experiments in rats were approved by the Ethics Committee on the Use of Animals of the Ribeirão Preto Campus of the University of Sao Paulo (CEUA)—(Protocol No. 14.1.479.53.0).

### Example 7—Analysis of the Ability of SEQ. ID. 17 and of SEQ. ID. 19 to Induce Increased Ovarian Mass in Rats

The assessment of the ability of SEQ. ID. 17 and SEQ. ID. 19 to promote activity in vivo related to the hormonal function of chorionic gonadotrophin was analyzed by the measurement of the ovarian mass of rats treated with SEQ. ID. 17 [r-eCG without GFP (10 up)] and SEQ. ID. 19 [r-eCG with GFP (20 ug)]. The effects of SEQ. ID. 17 on ovarian growth induction were then evaluated by measuring the ovarian mass of rats (Wistar) at 21 days after intramuscular injection of 10 ug of SEQ. ID. 17 (FIG. 8). Each experimental group contained 4 animals (total of 8 ovaries per group). The procedures used in the induction experiments of ovarian mass increase in rats were approved by CEUA (Protocol No. 14.1.479.53.0).

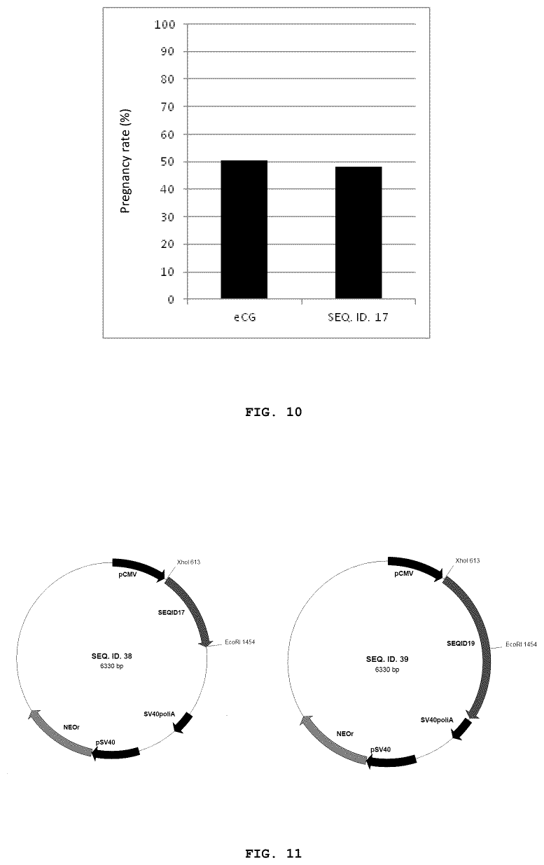

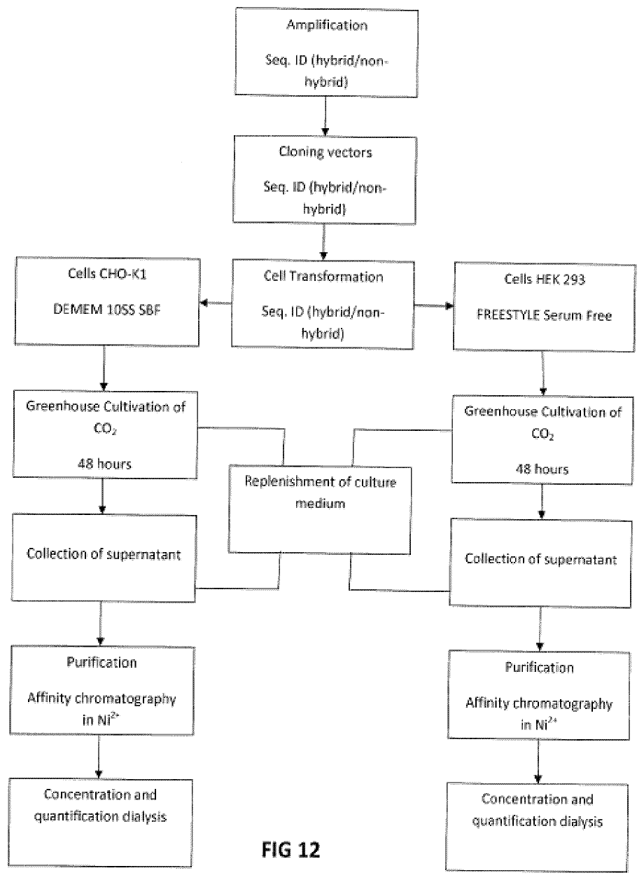

Likewise, FIG. 9 shows the functional analysis (induction of ovarian mass increase) comparative between the recombinant forms of Green Fluorescent Protein (GFP, SEQ. ID. 49), the native form of eCG (SEQ. ID. 5 and SEQ. ID. 7) and of SEQ. ID. 19. Each experimental group contained 4 animals (total of 8 ovaries per group) and only 2 ovaries from each experimental group, randomized, are represented. The scale associated with the images is dimensioned in centimeters.

The results are expressed as ovarian mass in grams (g) and indicate that the recombinant forms of eCG (SEQ. ID. 17 and SEQ. ID. 19) exhibit in vivo bioactivity similar to native eCG (SEQ. ID. 5 and SEQ. ID. 7). These examples aid in the rationale for including hybrid forms of the hormone (composed of the alpha chain of equine and target animals) in this patent, since SEQ. ID. 17 and 19 show genetic similarity above 97% and structural similarities with the hybrid forms, which may be indicated for use in several species of mammals.

### Example 8—Field Tests for the Evaluation of the Activity of SEQ. ID. 17 in Large Animals (Cattle)

The hormonal activity of SEQ. ID. 17 was evaluated in estrus synchronization protocols (IATF) in large mammals (Bovine) by Ultrasound (GE-Logiq and Transrectal transducer mod I-739, 8-12 Mhz) in females induced to estrus through hormonal protocols carried out by administration of eCG 300 IU and SEQ. ID. 17 30 μg.

The experimental model was based on Bos taurus indicus females, in homogeneous groups for the animal category (race, age, calving at least 1 time), with n=127 for the eCG group and n=50 for the SEQ ID. 17 group. The signs of estrus were verified by clinical evaluation and by comparison of follicular waves and ovulation (ultrasound).

The IATF protocol consisted of the introduction of the vaginal device for progesterone release (day 1), administration of eCG 300 IU and SEQ. ID. 17 30 μg and uterine evaluation by ultrasonography (day 8). Insemination was performed after the analysis by ultrasonography and follicular wave observation, where at least one follicle presented growth (1.4 mm/day) for each animal of both groups (day 10).

FIG. 10 shows comparison of pregnancy rate which was performed 30 days post-insemination by trans-rectal ultrasonography in animals belonging to the inseminated groups from the IATF protocol using the eCG and SEQ. ID. 17. The results obtained showed a pregnancy rate of 50.23% for eCG, and of 48% for SEQ. ID. 17, where the national average pregnancy rate per IATF is 42%.

The recombinant eCG were also assayed on animals of commercial interest such as cattle and pigs.

### a) Cattle

30 and 40 μg of rCG was applied to in anesthro cows, in order to study its ability to induce follicle growth and the pregnancy in cows by fixed-time artificial insemination (FTAI). The study in cows was carried out in two commercial farms, located in Alfenas, Minas Gerais, Brazil, with 20 animals (Nelore—Bos indicus) per group, in 4 groups, totaling 80 animals. The standard reproduction protocol was used, which consisted of the insertion (day 0) and removal (day 8) of an intravaginal device that releases progesterone; two injections of estradiol benzoate (day 0 and day 9); an injection of prostaglandin (day 8) and an injection with one of two different doses of reCG (30 and 40 μg), PMSG (positive control) and saline (negative control) on day 8.

From day 8, after the removal of the intravaginal progesterone device, the estrous signals were checked twice daily until day 10. Fixed-time artificial insemination (FTAI) was performed 30 hours after the second injection of estradiol (54 hours after the removal of the progesterone device). Ultrasound (transrectal) was performed between Days 8 and 12, twice daily, to measure follicular dynamics. On days 40 and 70, an ultrasound was performed to identify the pregnant cows. At the end of the study, all the cows were released to the herd. Estrus monitoring between day 8 and 10 showed a significant difference between the control group D (negative control) when compared to the treated groups A (30 μg), B (40 μg), and C (positive control) (P<0.05). Only 2 of the 20 animals in the control group had estrus detected, while 9 animals from group A, 9 cows from group B and 10 animals from group C had estrus.

The measurement of the dominant follicle for each animal was performed on days 8 and 10 of the treatment, the average size of the follicle in the first measurement was 9.36 mm, and in the second one it was 11.24 mm.

All 80 cows (20 per group) were inseminated after standard treatment. On days 40 and 70 after the beginning of treatment, the diagnosis of pregnancy was performed. The results showed that the groups with administration of rCG had higher pregnancies than the negative control. In conclusion, the reCG was able to induce pregnancy and stimulate the animals in a assisted reproduction technique.

### b) Pigs

In vivo test in pigs to evaluate the biological activity of rCG were also performed, combined also with hCG (Chorulon-MSD) to induce puberty, fertility and reproduction in females mated to the induced estrus. This study was conducted at the Federal Institute of Education, Science and Technology of the south of Minas Gerais—Campus Muzambinho, Minas Gerais, Brazil. The prepubertal females (n=18 per group) were injected intramuscularly with a dose of reCG combined with a dose of Chorulon or injected with PG® 600 (positive control) or vehicle (negative control). After the estrus treatment signals, they were checked twice daily from day 3 to day 7 and artificial insemination (AI) was performed based on heat detection. Blood samples were collected before dosing to confirm the anovulatory state and after administration to evaluate the changes in progesterone concentrations. On day 31, ultrasound was performed to identify pregnant females. At the end of the experiment (days 33 to 37), pregnant animals were sacrificed to perform a postmortem macroscopic examination of the reproductive tract.

The number of sows exhibiting estrous signals showed no statistical difference between the groups. Although the groups treated with reCG+Chorulon and PG 600 have exhibited estrus signals before the saline treated animals, the animals treated with saline solution had no influence on the treatment, but repeated exposure to the male eventually induced puberty in some animals. The animals that had estrous detected on days 3, 4 and 5 are more likely to be influenced by the treatment.

The applicant concluded that it was possible to produce homogeneous and active preparations of reCG in laboratory scale through a cell culture biotechnological process. The results showed that the recombinant CG may be used in assisted reproductive techniques in large animals in vivo, even the origin of the sequence is of a different species.
Recombinant hybrid CGs have also effect in other mammals. The rCG for E. caballus (SEQ ID NO: 17), B. taurus (SEQ ID NO: 23), S. scrofa (SEQ ID NO: 27), O. aries (SEQ ID NO: 31) and C. hircus (SEQ ID NO: 35) were tested in order to assess whether these hormones were able to promote in vivo activity related to the hormonal function of chorionic gonadotropin. The ovarian mass of rats treated with an intramuscular injection of 20 μg of each rCG was measure in rats (Wistar) at 21 days of life. Each experimental group contained 04 animals (total of 8 ovaries per group). The procedures used in experiments to increase ovarian mass in rats were approved by CEUA (Protocol No. 14.1.479.53.0).

The figure below shows the result of this experiment. As it may be seen, all rCG produce a significant increase in the ovarian mass, despite being from a different genus. The recombinant hormones claimed have in vivo effect in mammals, which make these molecules excellent candidates in non-human fertility treatments.

The analyzes showed the formation of cysts in 5.5% and twin formation in 1.59% in the eCG group, where the group SEQ. ID. 17 did not present the formation of cysts and twins.

Applications

From an effective amount of the recombinant glycoprotein hormones of SEQ. ID. 17 and SEQ. ID. 19, for example, from 0.001 to 10,000 μg, together with pharmaceutically acceptable adjuvants, such as hormone scavengers or permeants and Nanotechnology-based Release Systems, it is possible to propose a pharmaceutical composition. These adjuvants aim to ensure pharmacokinetic and pharmacodynamics quality by ensuring the adequate bioactivity of these recombinant hormones in different animal reproduction protocols. Such composition is used for assisted animal reproduction comprising an effective amount of recombinant glycoprotein hormones. Such compositions will be used in induction of ovulation; induction of superovulation; follicular growth; induction of estrus; reversal of anestrous; induction of puberty in animals of commercial interest or not, mammals in general, such as cattle, sheep, goats, pigs, horses, buffaloes, bison, antelopes, domestic and wild species of canines and felines, cetaceans, ursids and primates.

In addition, there is also the possibility of elaborating kits for induction of ovulation; induction of superovulation; follicular growth; induction of estrus; reversal of anestrous; induction of puberty; for use in IATF protocols (Fixed Time Artificial Insemination) FIC (In vitro Fertilization), TETF (Fixed Time Embryo Transfer) in animals of commercial interest or not.

Considering that chorionic gonadotrophins can be immunogenic (induce antibody production) and antigenic (recognized by antibodies) (Herve et al. 2004, Forcada et al. 2011; Chopineau et al., 1993) it is possible to propose that the recombinant glycoprotein hormones in question can be used to obtain native (monoclonal and/or polyclonal) or recombinant (phage display) antibodies and that both these antibodies and recombinant glycoprotein hormones, and their derivatives (conjugates to enzymes, radiolabels and/or fluorochromes), may comprise kits for the detection of these two categories of molecules (hormones and anti-hormones) in biological samples or not.

Although the invention has been widely described, one person skilled in the art would find obvious that many changes and modifications may be made without covering said modifications by the scope of the invention.

### REFERENCES

Bousfield G R, Butnev V Y, Butnev V Y, Nguyen V T, Gray C M, Dias J A, MacColi R, Eisele L, Harvey Dj. Differential effects of alpha subunit Asparagine56 oligosaccharide structure on equine lutropin and follitropin hybrid conformation and receptor-binding activity. Biochemistry; 43(33):10817-10833, 2004.
Chopineau M, Maurel M C, Combarnous' Y, Durand P. Topography of equine chorionic gonadotropin epitopes relative to the luteinizing hormone and follicle-stimulating hormone receptor interaction sites. Mol Cell Endocrinol, 92(2):229-239, 1993.
Forcada F, Ait Amer-Meziane M, Abecia J A, Maurel M C, Cebrian-Pérez J A, Muiho-Blanco T, Asenjo B, Vazquez Ml, Casao A. Repeated superovulation using a simplified FSH/eCG treatment for in vivo embryo production in sheep. Theriogenology, 75(4)769-776, 2011.
Galet C, Guillou F, Foulon-Gauze F, Combarnous Y, Chopineau M. The beta104-109 sequence is essential for the secretion of correctly folded single-chain beta alpha horse L H/C G and for its FSH activity. J Endocrinol, 203(1): 167-174, 2009.
Gopalkrishnan R V, Christiansen K A, Goldstein N I, DePinho R A, Fisher P B. Use of the human EF-1 alpha promoter for expression can significantly increase success in establishing stable cell lines with consistent expression: a study using the tetracycline-inducible system in human cancer cells. Nucleic Acids Res. December 15; 27(24): 4775-82. 1999.
Hervé V, Roy F, Bertin J, Guillou F, Maurel M C. Antiequine chorionicgonadotropin (eCG) antibodies generated in goats treated with eCG for the induction of ovulation modulate the luteinizing hormone and follicle-stimulating hormone bioactivities of eCG differently. Endocrinology, 145(1):294-303, 2004.
Min K S, Hattori N, Aikawa J, Shiota K, Ogawa T. Site-directed mutagenesis of recombinant equine chorionic gonadotropin/luteinizing hormone: differential role of oligosaccharides in luteinizing hormone- and follicle-stimulating hormone-like activities. Endocr J 43(5):585-593, 1996. Min K S, Hiyama T, Seong H H, Hattori N, Tanaka S, Shiota K. Biological activities of tethered equine chorionic gonadotropin (eCG) and its deglycosylated mutants. J Reprod Dev, 50(3):297-304, 2004.
Moritz B, Becker P B, Gopfert U. CMV promoter mutants with a reduced propensity to productivity loss in CHO cells. Sei Rep. November 19; 5: 16952. doi: 10.1038/srep16952. 2015.
Moyle W R, Campbell R K, Myers R V, Bernard M P, Han Y, Wang X. Coevolution of ligand-receptor pairs. Nature, 368(6468): 251-255, 1994.
Mullis K, Faloona F, ScharfS, Saiki R, Horn G, Erlich H. Specific enzymatic amplification of DNA in vitro: the polymerase chain reaction. Cold Spring Harb Symp Quant Biol.; 51 Pt 1: 263-73. 1986.
Murphy, B D. Equine chorionic gonadotropin: an enigmatic but essential tool. Anim Reprod, 9 (3): 223-230, 2012.
Sambrook, J.; Fritsch, E. F.; Maniatis, T: Molecular Cloning: A laboratory manual. 2nd ed. Cold Spring Harbor Laboratory Press, Cold Spring Harbor, N. Y. 1989. Sanger, F., Nicklen, S. And Coulson, A R. DNA sequencing with chain terminating inhibitors. Proc. Natl. Acad. Sci. USA, 74:5463-5467. 1977.

1. A process for the production and purification of a hybrid chorionic gonadotropin, comprising the steps of:
(a) amplification, modification and cloning an DNA molecule encoding a polypeptide selected from the group consisting of SEQ NO:23, SEQ NO: 25, SEQ NO: 27, SEQ NO: 29, SEQ NO:31, SEQ NO:33, SEQ NO:35 and SEQ NO: 37;
(b) construction of the expression vectors comprising the sequences of step (a);
(c) transfection of a human cell line with the vector of step (b), expression and analysis of cells;
(d) purification of the hybrid chorionic gonadotropin by affinity chromatography; and
(e) dialysis and sterilization of the hybrid chorionic gonadotropin obtained in step (d).

2. The process of claim 1 wherein the DNA in step (a) is selected from the group consisting of SEQ NO: 22, SEQ NO: 24, SEQ NO: 26, SEQ NO: 28, SEQ NO: 30, SEQ NO: 32, SEQ NO: 34 and SEQ NO: 36.

3. The process according to claim 1, wherein the hybrid chorionic gonadotropin obtained in step (d) is selected from the group consisting of SEQ NO: 23, SEQ NO: 25, SEQ NO: 27, SEQ NO: 29, SEQ NO: 31, SEQ NO: 33, SEQ NO: 35 and SEQ NO: 37.

4. A recombinant hybrid chorionic gonadotropin produced by the process as defined in claim 1, comprising the subunit 13 of the equine chorionic gonadotropin of sequence SEQ NO: 7 and a subunit a selected from the group consisting of SEQ NO: 9, SEQ NO: 11, SEQ NO: 13 and SEQ NO: 15, a purification marker, secretion signaling peptide of the molecule, a dimerization interface peptide, a specific proteolytic site and optionally, a fluorescent label.

5. The recombinant hybrid chorionic gonadotropin of claim 4, wherein the recombinant hybrid chorionic gonadotropin is selected from the group consisting of SEQ NO: 23, SEQ NO: 25, SEQ NO: 27, SEQ NO: 29, SEQ NO: 31, SEQ NO: 33, SEQ NO: 35 and SEQ NO: 37.

6. A method for assisted animal reproduction technique in a mammal in need of same, comprising administering the recombinant hybrid chorionic gonadotropin produced by the process of claim 1 to the mammal.

7. The method according to claim 6, wherein the mammal is selected from the group consisting of cattle, sheep, goats, swine, horses, mules, bubalins, bison, antelopes, domestic and wild species of canines and felines, cetaceans, ursids and primates.

8. The method according to claim 7, wherein the mammal is cattle.

9. The method according to claim 6, wherein the assisted animal reproduction technique is selected from the group consisting of: ovulation induction, induction of superovulation, follicular growth, induction of estrus, reversal of anestrous, puberty induction, use in FTAI protocols (Fixed-Time Artificial Insemination), IVF (in vitro fertilization) protocols and FTET protocols (Fixed Time Embryo Transfer).

10. The method according to claim 6, wherein the recombinant chorionic gonadotropin is administered in an amount of 0.001 to 10,000 119, observing the body weight of the target animal.

11. A pharmaceutical composition comprising a recombinant chorionic gonadotropin as defined in claim 4, and a carrier therefor.

12. An expression vector of recombinant glycoprotein hormones comprising an ONA sequence of a hybrid chorionic gonadotropin, wherein the sequence of the expression vector is selected from the group consisting of SEQ NO: 4, SEQ NO: 41, SEQ NO: 42, SEQ NO: 43, SEQ NO: 44, SEQ NO: 45, SEQ NO: 46 and SEQ NO: 47.

13. The process according to claim 2, wherein the hybrid chorionic gonadotropin obtained in step (d) is selected from the group consisting of SEQ NO: 23, SEQ NO: 25, SEQ NO: 27, SEQ NO: 29, SEQ NO: 31, SEQ NO: 33, SEQ NO: 35 and SEQ NO: 37.

14. A recombinant hybrid chorionic gonadotropin produced by the process as defined in claim 2, comprising the subunit 13 of the equine chorionic gonadotropin of sequence SEQ NO: 7 and a subunit a selected from the group consisting of SEQ NO: 9, SEQ NO: 11, SEQ NO: 13 and SEQ NO: 15, a purification marker, secretion signaling peptide of the molecule, a dimerization interface peptide, a specific proteolytic site and optionally, a fluorescent label.

15. A pharmaceutical composition comprising a recombinant chorionic gonadotropin as defined in claim 5, and a carrier therefor.


SEQUENCE LISTING (49 sequences)

SEQ ID NO: 1 - DNA, 42 bp, unidentified
  misc_feature 1..42: note = Sequence of nucleotide primers of the different forms of CG
  source 1..42: mol_type = unassigned DNA
ctcgagatgcatcatcatcatcatcatgagacgctccagggg

SEQ ID NO: 2 - DNA, 50 bp, unidentified
  misc_feature 1..50: note = Sequence of nucleotide primers of the different forms of CG
  source 1..50: mol_type = unassigned DNA
gaattcgggattggaagtacaggttctcaatcttgtggtgatagcaagtg

SEQ ID NO: 3 - DNA, 50 bp, unidentified
  misc_feature 1..50: note = Sequence of nucleotide primers of the different forms of CG
  source 1..50: mol_type = unassigned DNA
gaattcgttaggattggaagtacaggttctcaatcttgtggtgatagcaa

SEQ ID NO: 4 - DNA, 363 bp, unidentified
  misc_feature 1..363: note = Native eCG: Alpha subunit
  source 1..363: mol_type = unassigned DNA
atggattactacagaaaacatgcagctgtcatcctggccacattgtccgtgtttctgcatattctccattcctttcctgatggagagtttacaacgcaggattgcccagaatgcaagctaagggaaaacaagtacttcttcaaactgggcgtcccgatttaccagtgtaagggctgctgcttctccagagcgtaccccactccagcaaggtccaggaagacaatgttggtcccaaagaacatcacctcagaatccacatgctgtgtggccaaagcatttatcagggtcacagtgatgggaaacatcaagttggagaaccacacccagtgctattgcagcacttgctatcaccacaagatttaa

SEQ ID NO: 5 - protein, 120 aa, unidentified
  REGION 1..120: note = Native eCG: Alpha subunit
MDYYRKHAAVILATLSVFLHILHSFPDGEFTTQDCPECKLRENKYFFKLGVPIYQCKGCCFSRAYPTPARSRKTMLVPKNITSESTCCVAKAFIRVTVMGNIKLENHTQCYCSTCYHHKI

SEQ ID NO: 6 - DNA, 510 bp, unidentified
  misc_feature 1..510: note = Native eCG: Beta subunit
  source 1..510: mol_type = unassigned DNA
atggagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcttga

SEQ ID NO: 7 - protein, 169 aa, unidentified
  REGION 1..169: note = Native eCG: Beta subunit
METLQGLLLWMLLSVGGVWASRGPLRPLCRPINATLAAEKEACPICITFTTSICAGYCPSMVRVMPAALPAIPQPVCTYRELRFASIRLPGCPPGVDPMVSFPVALSCHCGPCQIKTTDCGVFRDQPLACAPQASSSSKDPPSQPLTSTSTPTPGASRRSSHPLPIKTS

SEQ ID NO: 8 - DNA, 363 bp, unidentified
  misc_feature 1..363: note = Native bCG: Alpha subunit
  source 1..363: mol_type = unassigned DNA
atggattactacagaaaatatgcagctgtcattctggccattttgtctctgtttctgcaaattctccattcctttcctgatggagagtttacaatgcagggctgtcctgaatgcaagctaaaagaaaacaaatacttctccaagccagatgctccaatctatcagtgcatggggtgctgcttctccagggcataccccactccagcgaggtctaagaagacaatgttggtccccaagaacatcacctcggaagctacatgctgtgtggccaaagcatttaccaaggccacagtgatgggaaatgtcagagtggagaaccacaccgagtgccactgcagcacttgttattatcacaaatcctaa

SEQ ID NO: 9 - protein, 120 aa, unidentified
  REGION 1..120: note = Native bCG: Alpha subunit
MDYYRKYAAVILAILSLFLQILHSFPDGEFTMQGCPECKLKENKYFSKPDAPIYQCMGCCFSRAYPTPARSKKTMLVPKNITSEATCCVAKAFTKATVMGNVRVENHTECHCSTCYYHKS

SEQ ID NO: 10 - DNA, 363 bp, unidentified
  misc_feature 1..363: note = Native sCG: Alpha subunit
  source 1..363: mol_type = unassigned DNA
atggattactacagaaaatatgcagctgtcatcctggccatattgtctgtgtttctgcaaattctccattcctttcctgatggagagtttacaatgcagggctgcccagaatgcaagctaaaggaaaacaagtacttctccaagctgggtgccccaatctatcagtgcatgggctgctgcttctccagagcgtacccaactccagcgaggtccaagaagacaatgttggttccaaagaacatcacctcggaagccacatgctgtgtggccaaagcatttaccaaggccacagtaatgggaaatgccagagtggagaaccacaccgaatgccactgcagtacttgttattatcacaaatcttaa

SEQ ID NO: 11 - protein, 120 aa, unidentified
  REGION 1..120: note = Native sCG: Alpha subunit
MDYYRKYAAVILAILSVFLQILHSFPDGEFTMQGCPECKLKENKYFSKLGAPIYQCMGCCFSRAYPTPARSKKTMLVPKNITSEATCCVAKAFTKATVMGNARVENHTECHCSTCYYHKS

SEQ ID NO: 12 - DNA, 363 bp, unidentified
  misc_feature 1..363: note = Native oCG: Alpha subunit
  source 1..363: mol_type = unassigned DNA
atggattactacagaaagtatgcagctgccattctggccatattgtctctgtttctgcaaattctccattcctttcctgatggagagtttacaatgcagggttgtcctgaatgcaagctaaaagaaaacaaatacttctccaagccagatgctccaatttatcagtgcatggggtgctgcttctccagggcataccccactccagcgaggtctaagaagacaatgttggttcccaagaacatcacctcggaagccacatgttgtgtggccaaagcatttaccaaggccacagtgatgggaaatgtcagagtggagaaccacaccgagtgccactgcagtacttgttattatcacaaatcttaa

SEQ ID NO: 13 - protein, 120 aa, unidentified
  REGION 1..120: note = Native oCG: Alpha subunit
MDYYRKYAAAILAILSLFLQILHSFPDGEFTMQGCPECKLKENKYFSKPDAPIYQCMGCCFSRAYPTPARSKKTMLVPKNITSEATCCVAKAFTKATVMGNVRVENHTECHCSTCYYHKS

SEQ ID NO: 14 - DNA, 363 bp, unidentified
  misc_feature 1..363: note = Native cCG: Alpha subunit
  source 1..363: mol_type = unassigned DNA
atggattactacagaaaatatgcagctgtcattctggccgtattgtctctgttcctgcagatactccattcctttcctgatggagagtttatgatgcagggttgtcctgaatgcaagctaaaggaaaacaaatacttctccaagccagacgctccaatctatcagtgcatgggctgctgcttctccagggcataccccactccagcgaggtctaagaagacaatgttggtccccaagaacatcacctcggaagccacatgctgtgtggccaaagcgtttaccaaggccacagtgacgggaaacgtcagagtggagaaccacaccgactgccactgcagtacttgttattatcacaaatcttaa

SEQ ID NO: 15 - protein, 120 aa, unidentified
  REGION 1..120: note = Native cCG: Alpha subunit
MDYYRKYAAVILAVLSLFLQILHSFPDGEFMMQGCPECKLKENKYFSKPDAPIYQCMGCCFSRAYPTPARSKKTMLVPKNITSEATCCVAKAFTKATVTGNVRVENHTDCHCSTCYYHKS

SEQ ID NO: 16 - DNA, 816 bp, unidentified
  misc_feature 1..816: note = r-eCG
  source 1..816: mol_type = unassigned DNA
atgcatcatcatcatcatcatgagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcttttcctgatggagagtttacaacgcaggattgcccagaatgcaagctaagggaaaacaagtacttcttcaaactgggcgtcccgatttaccagtgtaagggctgctgcttctccagagcgtaccccactccagcaaggtccaggaagacaatgttggtcccaaagaacatcacctcagaatccacatgctgtgtggccaaagcatttatcagggtcacagtgatgggaaacatcaagttggagaaccacacccagtgctattgcagcacttgctatcaccacaagatttaa

SEQ ID NO: 17 - protein, 271 aa, unidentified
  REGION 1..271: note = r-eCG
MHHHHHHETLQGLLLWMLLSVGGVWASRGPLRPLCRPINATLAAEKEACPICITFTTSICAGYCPSMVRVMPAALPAIPQPVCTYRELRFASIRLPGCPPGVDPMVSFPVALSCHCGPCQIKTTDCGVFRDQPLACAPQASSSSKDPPSQPLTSTSTPTPGASRRSSHPLPIKTSFPDGEFTTQDCPECKLRENKYFFKLGVPIYQCKGCCFSRAYPTPARSRKTMLVPKNITSESTCCVAKAFIRVTVMGNIKLENHTQCYCSTCYHHKI

SEQ ID NO: 18 - DNA, 1605 bp, unidentified
  misc_feature 1..1605: note = r-eCG-GFP
  source 1..1605: mol_type = unassigned DNA
atgcatcatcatcatcatcatgagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcttttcctgatggagagtttacaacgcaggattgcccagaatgcaagctaagggaaaacaagtacttcttcaaactgggcgtcccgatttaccagtgtaagggctgctgcttctccagagcgtaccccactccagcaaggtccaggaagacaatgttggtcccaaagaacatcacctcagaatccacatgctgtgtggccaaagcatttatcagggtcacagtgatgggaaacatcaagttggagaaccacacccagtgctattgcagcacttgctatcaccacaagattgagaacctgtacttccaatcccgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 19 - protein, 534 aa, unidentified
  REGION 1..534: note = r-eCG-GFP
MHHHHHHETLQGLLLWMLLSVGGVWASRGPLRPLCRPINATLAAEKEACPICITFTTSICAGYCPSMVRVMPAALPAIPQPVCTYRELRFASIRLPGCPPGVDPMVSFPVALSCHCGPCQIKTTDCGVFRDQPLACAPQASSSSKDPPSQPLTSTSTPTPGASRRSSHPLPIKTSFPDGEFTTQDCPECKLRENKYFFKLGVPIYQCKGCCFSRAYPTPARSRKTMLVPKNITSESTCCVAKAFIRVTVMGNIKLENHTQCYCSTCYHHKIENLYFQSRILQSTVPRARDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 20 - DNA, 59 bp, unidentified
  misc_feature 1..59: note = Export sequence
  source 1..59: mol_type = unassigned DNA
gagacgctccaggggctgctgctgtggatgctgctgagtgttggcggggtctgggcatc

SEQ ID NO: 21 - protein, 20 aa, unidentified
  REGION 1..20: note = Export sequence
ETLQGLLLWMLLSVGGVWAS

SEQ ID NO: 22 - DNA, 798 bp, unidentified
  misc_feature 1..798: note = r-beCG
  source 1..798: mol_type = unassigned DNA
atggagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcttttcctgatggagagtttacaatgcagggctgtcctgaatgcaagctaaaagaaaacaaatacttctccaagccagatgctccaatctatcagtgcatggggtgctgcttctccagggcataccccactccagcgaggtctaagaagacaatgttggtccccaagaacatcacctcggaagctacatgctgtgtggccaaagcatttaccaaggccacagtgatgggaaatgtcagagtggagaaccacaccgagtgccactgcagcacttgttattatcacaaatcctaa

SEQ ID NO: 23 - protein, 265 aa, unidentified
  REGION 1..265: note = r-beCG
METLQGLLLWMLLSVGGVWASRGPLRPLCRPINATLAAEKEACPICITFTTSICAGYCPSMVRVMPAALPAIPQPVCTYRELRFASIRLPGCPPGVDPMVSFPVALSCHCGPCQIKTTDCGVFRDQPLACAPQASSSSKDPPSQPLTSTSTPTPGASRRSSHPLPIKTSFPDGEFTMQGCPECKLKENKYFSKPDAPIYQCMGCCFSRAYPTPARSKKTMLVPKNITSEATCCVAKAFTKATVMGNVRVENHTECHCSTCYYHKS

SEQ ID NO: 24 - DNA, 1605 bp, unidentified
  misc_feature 1..1605: note = r-beCG-GFP
  source 1..1605: mol_type = unassigned DNA
atgcatcatcatcatcatcatgagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcttttcctgatggagagtttacaatgcagggctgtcctgaatgcaagctaaaagaaaacaaatacttctccaagccagatgctccaatctatcagtgcatggggtgctgcttctccagggcataccccactccagcgaggtctaagaagacaatgttggtccccaagaacatcacctcggaagctacatgctgtgtggccaaagcatttaccaaggccacagtgatgggaaatgtcagagtggagaaccacaccgagtgccactgcagcacttgttattatcacaaatccgagaacctgtacttccaatcccgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 25 - protein, 534 aa, unidentified
  REGION 1..534: note = r-beCG-GFP
MHHHHHHETLQGLLLWMLLSVGGVWASRGPLRPLCRPINATLAAEKEACPICITFTTSICAGYCPSMVRVMPAALPAIPQPVCTYRELRFASIRLPGCPPGVDPMVSFPVALSCHCGPCQIKTTDCGVFRDQPLACAPQASSSSKDPPSQPLTSTSTPTPGASRRSSHPLPIKTSFPDGEFTMQGCPECKLKENKYFSKPDAPIYQCMGCCFSRAYPTPARSKKTMLVPKNITSEATCCVAKAFTKATVMGNVRVENHTECHCSTCYYHKSENLYFQSRILQSTVPRARDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 26 - DNA, 816 bp, unidentified
  misc_feature 1..816: note = r-seCG
  source 1..816: mol_type = unassigned DNA
atgcatcatcatcatcatcatgagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcctttcctgatggagagtttacaatgcagggctgcccagaatgcaagctaaaggaaaacaagtacttctccaagctgggtgccccaatctatcagtgcatgggctgctgcttctccagagcgtacccaactccagcgaggtccaagaagacaatgttggttccaaagaacatcacctcggaagccacatgctgtgtggccaaagcatttaccaaggccacagtaatgggaaatgccagagtggagaaccacaccgaatgccactgcagtacttgttattatcacaaatcttaa

SEQ ID NO: 27 - protein, 271 aa, unidentified
  REGION 1..271: note = r-seCG
MHHHHHHETLQGLLLWMLLSVGGVWASRGPLRPLCRPINATLAAEKEACPICITFTTSICAGYCPSMVRVMPAALPAIPQPVCTYRELRFASIRLPGCPPGVDPMVSFPVALSCHCGPCQIKTTDCGVFRDQPLACAPQASSSSKDPPSQPLTSTSTPTPGASRRSSHPLPIKTSFPDGEFTMQGCPECKLKENKYFSKLGAPIYQCMGCCFSRAYPTPARSKKTMLVPKNITSEATCCVAKAFTKATVMGNARVENHTECHCSTCYYHKS

SEQ ID NO: 28 - DNA, 1605 bp, unidentified
  misc_feature 1..1605: note = r-seCG-GFP
  source 1..1605: mol_type = unassigned DNA
atgcatcatcatcatcatcatgagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcctttcctgatggagagtttacaatgcagggctgcccagaatgcaagctaaaggaaaacaagtacttctccaagctgggtgccccaatctatcagtgcatgggctgctgcttctccagagcgtacccaactccagcgaggtccaagaagacaatgttggttccaaagaacatcacctcggaagccacatgctgtgtggccaaagcatttaccaaggccacagtaatgggaaatgccagagtggagaaccacaccgaatgccactgcagtacttgttattatcacaaatctgagaacctgtacttccaatcccgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 29 - protein, 534 aa, unidentified
  REGION 1..534: note = r-seCG-GFP
MHHHHHHETLQGLLLWMLLSVGGVWASRGPLRPLCRPINATLAAEKEACPICITFTTSICAGYCPSMVRVMPAALPAIPQPVCTYRELRFASIRLPGCPPGVDPMVSFPVALSCHCGPCQIKTTDCGVFRDQPLACAPQASSSSKDPPSQPLTSTSTPTPGASRRSSHPLPIKTSFPDGEFTMQGCPECKLKENKYFSKLGAPIYQCMGCCFSRAYPTPARSKKTMLVPKNITSEATCCVAKAFTKATVMGNARVENHTECHCSTCYYHKSENLYFQSRILQSTVPRARDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 30 - DNA, 816 bp, unidentified
  misc_feature 1..816: note = r-oeCG
  source 1..816: mol_type = unassigned DNA
atgcatcatcatcatcatcatgagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcctttcctgatggagagtttacaatgcagggttgtcctgaatgcaagctaaaagaaaacaaatacttctccaagccagatgctccaatttatcagtgcatggggtgctgcttctccagggcataccccactccagcgaggtctaagaagacaatgttggttcccaagaacatcacctcggaagccacatgttgtgtggccaaagcatttaccaaggccacagtgatgggaaatgtcagagtggagaaccacaccgagtgccactgcagtacttgttattatcacaaatcttaa

SEQ ID NO: 31 - protein, 271 aa, unidentified
  REGION 1..271: note = r-oeCG
MHHHHHHETLQGLLLWMLLSVGGVWASRGPLRPLCRPINATLAAEKEACPICITFTTSICAGYCPSMVRVMPAALPAIPQPVCTYRELRFASIRLPGCPPGVDPMVSFPVALSCHCGPCQIKTTDCGVFRDQPLACAPQASSSSKDPPSQPLTSTSTPTPGASRRSSHPLPIKTSFPDGEFTMQGCPECKLKENKYFSKPDAPIYQCMGCCFSRAYPTPARSKKTMLVPKNITSEATCCVAKAFTKATVMGNVRVENHTECHCSTCYYHKS

SEQ ID NO: 32 - DNA, 1605 bp, unidentified
  misc_feature 1..1605: note = r-oeCG-GFP
  source 1..1605: mol_type = unassigned DNA
atgcatcatcatcatcatcatgagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcctttcctgatggagagtttacaatgcagggttgtcctgaatgcaagctaaaagaaaacaaatacttctccaagccagatgctccaatttatcagtgcatggggtgctgcttctccagggcataccccactccagcgaggtctaagaagacaatgttggttcccaagaacatcacctcggaagccacatgttgtgtggccaaagcatttaccaaggccacagtgatgggaaatgtcagagtggagaaccacaccgagtgccactgcagtacttgttattatcacaaatctgagaacctgtacttccaatcccgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 33 - protein, 534 aa, unidentified; identical to SEQ ID NO: 25
  REGION 1..534: note = r-oeCG-GFP

SEQ ID NO: 34 - DNA, 816 bp, unidentified
  misc_feature 1..816: note = r-ceCG
  source 1..816: mol_type = unassigned DNA
atgcatcatcatcatcatcatgagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcctttcctgatggagagtttatgatgcagggttgtcctgaatgcaagctaaaggaaaacaaatacttctccaagccagacgctccaatctatcagtgcatgggctgctgcttctccagggcataccccactccagcgaggtctaagaagacaatgttggtccccaagaacatcacctcggaagccacatgctgtgtggccaaagcgtttaccaaggccacagtgacgggaaacgtcagagtggagaaccacaccgactgccactgcagtacttgttattatcacaaatcttaa

SEQ ID NO: 35 - protein, 271 aa, unidentified
  REGION 1..271: note = r-ceCG
MHHHHHHETLQGLLLWMLLSVGGVWASRGPLRPLCRPINATLAAEKEACPICITFTTSICAGYCPSMVRVMPAALPAIPQPVCTYRELRFASIRLPGCPPGVDPMVSFPVALSCHCGPCQIKTTDCGVFRDQPLACAPQASSSSKDPPSQPLTSTSTPTPGASRRSSHPLPIKTSFPDGEFMMQGCPECKLKENKYFSKPDAPIYQCMGCCFSRAYPTPARSKKTMLVPKNITSEATCCVAKAFTKATVTGNVRVENHTDCHCSTCYYHKS

SEQ ID NO: 36 - DNA, 1605 bp, unidentified
  misc_feature 1..1605: note = r-ceCG-GFP
  source 1..1605: mol_type = unassigned DNA
atgcatcatcatcatcatcatgagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcctttcctgatggagagtttatgatgcagggttgtcctgaatgcaagctaaaggaaaacaaatacttctccaagccagacgctccaatctatcagtgcatgggctgctgcttctccagggcataccccactccagcgaggtctaagaagacaatgttggtccccaagaacatcacctcggaagccacatgctgtgtggccaaagcgtttaccaaggccacagtgacgggaaacgtcagagtggagaaccacaccgactgccactgcagtacttgttattatcacaaatctgagaacctgtacttccaatcccgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 37 - protein, 534 aa, unidentified
  REGION 1..534: note = r-ceCG-GFP
MHHHHHHETLQGLLLWMLLSVGGVWASRGPLRPLCRPINATLAAEKEACPICITFTTSICAGYCPSMVRVMPAALPAIPQPVCTYRELRFASIRLPGCPPGVDPMVSFPVALSCHCGPCQIKTTDCGVFRDQPLACAPQASSSSKDPPSQPLTSTSTPTPGASRRSSHPLPIKTSFPDGEFMMQGCPECKLKENKYFSKPDAPIYQCMGCCFSRAYPTPARSKKTMLVPKNITSEATCCVAKAFTKATVTGNVRVENHTDCHCSTCYYHKSENLYFQSRILQSTVPRARDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 38 - DNA, 5521 bp, unidentified
  misc_feature 1..5521: note = peCG
  source 1..5521: mol_type = unassigned DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagatggagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcttttcctgatggagagtttacaacgcaggattgcccagaatgcaagctaagggaaaacaagtacttcttcaaactgggcgtcccgatttaccagtgtaagggctgctgcttctccagagcgtaccccactccagcaaggtccaggaagacaatgttggtcccaaagaacatcacctcagaatccacatgctgtgtggccaaagcatttatcagggtcacagtgatgggaaacatcaagttggagaaccacacccagtgctattgcagcacttgctatcaccacaagatttaacgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 39 - DNA, 6329 bp, unidentified
  misc_feature 1..6329: note = peCG-GFP
  source 1..6329: mol_type = unassigned DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagatgcatcatcatcatcatcatgagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcttttcctgatggagagtttacaacgcaggattgcccagaatgcaagctaagggaaaacaagtacttcttcaaactgggcgtcccgatttaccagtgtaagggctgctgcttctccagagcgtaccccactccagcaaggtccaggaagacaatgttggtcccaaagaacatcacctcagaatccacatgctgtgtggccaaagcatttatcagggtcacagtgatgggaaacatcaagttggagaaccacacccagtgctattgcagcacttgctatcaccacaagattgagaacctgtacttccaatcccgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 40 - DNA, 5540 bp, unidentified
  misc_feature 1..5540: note = pbeCG
  source 1..5540: mol_type = unassigned DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagatgcatcatcatcatcatcatgagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcttttcctgatggagagtttacaatgcagggctgtcctgaatgcaagctaaaagaaaacaaatacttctccaagccagatgctccaatctatcagtgcatggggtgctgcttctccagggcataccccactccagcgaggtctaagaagacaatgttggtccccaagaacatcacctcggaagctacatgctgtgtggccaaagcatttaccaaggccacagtgatgggaaatgtcagagtggagaaccacaccgagtgccactgcagcacttgttattatcacaaatcctaacgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 41 - DNA, 5558 bp, unidentified
  misc_feature 1..5558: note = pbeCG-GFP
  source 1..5558: mol_type = unassigned DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagatgcatcatcatcatcatcatgagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcttttcctgatggagagtttacaatgcagggctgtcctgaatgcaagctaaaagaaaacaaatacttctccaagccagatgctccaatctatcagtgcatggggtgctgcttctccagggcataccccactccagcgaggtctaagaagacaatgttggtccccaagaacatcacctcggaagctacatgctgtgtggccaaagcatttaccaaggccacagtgatgggaaatgtcagagtggagaaccacaccgagtgccactgcagcacttgttattatcacaaatccgagaacctgtacttccaatcccgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 42 - DNA, 5540 bp, unidentified
  misc_feature 1..5540: note = pseCG
  source 1..5540: mol_type = unassigned DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagatgcatcatcatcatcatcatgagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcctttcctgatggagagtttacaatgcagggctgcccagaatgcaagctaaaggaaaacaagtacttctccaagctgggtgccccaatctatcagtgcatgggctgctgcttctccagagcgtacccaactccagcgaggtccaagaagacaatgttggttccaaagaacatcacctcggaagccacatgctgtgtggccaaagcatttaccaaggccacagtaatgggaaatgccagagtggagaaccacaccgaatgccactgcagtacttgttattatcacaaatcttaacgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 43 - DNA, 6329 bp, unidentified
  misc_feature 1..6329: note = pseCG-GFP
  source 1..6329: mol_type = unassigned DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagatgcatcatcatcatcatcatgagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcctttcctgatggagagtttacaatgcagggctgcccagaatgcaagctaaaggaaaacaagtacttctccaagctgggtgccccaatctatcagtgcatgggctgctgcttctccagagcgtacccaactccagcgaggtccaagaagacaatgttggttccaaagaacatcacctcggaagccacatgctgtgtggccaaagcatttaccaaggccacagtaatgggaaatgccagagtggagaaccacaccgaatgccactgcagtacttgttattatcacaaatctgagaacctgtacttccaatcccgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 44 - DNA, 5540 bp, unidentified
  misc_feature 1..5540: note = poeCG
  source 1..5540: mol_type = unassigned DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagatgcatcatcatcatcatcatgagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcctttcctgatggagagtttacaatgcagggttgtcctgaatgcaagctaaaagaaaacaaatacttctccaagccagatgctccaatttatcagtgcatggggtgctgcttctccagggcataccccactccagcgaggtctaagaagacaatgttggttcccaagaacatcacctcggaagccacatgttgtgtggccaaagcatttaccaaggccacagtgatgggaaatgtcagagtggagaaccacaccgagtgccactgcagtacttgttattatcacaaatcttaacgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 45 - DNA, 6329 bp, unidentified
  misc_feature 1..6329: note = poeCG-GFP
  source 1..6329: mol_type = unassigned DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagatgcatcatcatcatcatcatgagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcctttcctgatggagagtttacaatgcagggttgtcctgaatgcaagctaaaagaaaacaaatacttctccaagccagatgctccaatttatcagtgcatggggtgctgcttctccagggcataccccactccagcgaggtctaagaagacaatgttggttcccaagaacatcacctcggaagccacatgttgtgtggccaaagcatttaccaaggccacagtgatgggaaatgtcagagtggagaaccacaccgagtgccactgcagtacttgttattatcacaaatctgagaacctgtacttccaatcccgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 46 - DNA, 5540 bp, unidentified
  misc_feature 1..5540: note = pceCG
  source 1..5540: mol_type = unassigned DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagatgcatcatcatcatcatcatgagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcctttcctgatggagagtttatgatgcagggttgtcctgaatgcaagctaaaggaaaacaaatacttctccaagccagacgctccaatctatcagtgcatgggctgctgcttctccagggcataccccactccagcgaggtctaagaagacaatgttggtccccaagaacatcacctcggaagccacatgctgtgtggccaaagcgtttaccaaggccacagtgacgggaaacgtcagagtggagaaccacaccgactgccactgcagtacttgttattatcacaaatcttaacgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 47 - DNA, 6329 bp, unidentified
  misc_feature 1..6329: note = pceCG-GFP
  source 1..6329: mol_type = unassigned DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagatgcatcatcatcatcatcatgagacgctccaggggctgctgctgtggatgctgctgagtgttggcggggtctgggcatccagggggccactgcggccactgtgccggcccatcaacgccactctggctgctgagaaggaggcctgccccatctgcatcaccttcaccaccagcatctgtgccggctactgccccagcatggtgcgggtgatgccagctgccctgccggccattccccagccagtgtgcacctaccgtgagctgcgctttgcttccatccggctccccggctgcccgcctggtgtggaccccatggtctccttccccgtggccctcagttgtcactgcgggccctgccagatcaagaccactgactgcggggttttcagagaccagcccttggcctgtgccccccaggcctcctcttcctctaaggatcccccatcccaacctctcacatccacatccaccccaactcctggggccagcagacgttcctctcatcccctcccaataaagacttcctttcctgatggagagtttatgatgcagggttgtcctgaatgcaagctaaaggaaaacaaatacttctccaagccagacgctccaatctatcagtgcatgggctgctgcttctccagggcataccccactccagcgaggtctaagaagacaatgttggtccccaagaacatcacctcggaagccacatgctgtgtggccaaagcgtttaccaaggccacagtgacgggaaacgtcagagtggagaaccacaccgactgccactgcagtacttgttattatcacaaatctgagaacctgtacttccaatcccgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 48 - DNA, 4733 bp, unidentified
  misc_feature 1..4733: note = preGFP
  source 1..4733: mol_type = unassigned DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 49 - protein, 239 aa, unidentified
  REGION 1..239: note = preGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK